(19) United States
(12) Patent Application Publication
(10) Pub. No.: US 2002/0169444 A1
(43) Pub. Date: Nov. 14, 2002

(54) Catheter having continuous braided electrode

(76) Inventors: Robert A. Mest, Long Beach, CA (US); Irma Hill, La Verne, CA (US)

Correspondence Address: CHRISTIE, PARKER & HALE, LLP, 350 WEST COLORADO BOULEVARD, SUITE 500, PASADENA, CA, 91105, US

(21) Appl. No.: 09/796,198

(22) Filed: May 8, 2001

(51) Int. Cl. A61B 18/18

(52) U.S. Cl. 606/41; 606/39

(57) ABSTRACT

A catheter for ablating tissue comprises an elongated flexible catheter body having a continuous electrode comprising a braided conductive mesh surrounding a flexible plastic tubing. In a preferred embodiment, the catheter comprises a catheter body, a tip section at the distal end of the catheter body, and an electrode assembly at the distal end of the tip section. The electrode assembly comprises a flexible plastic tubing having an outer wall with a plurality of irrigation holes extending therethrough, a generally circular main region that is generally transverse to the axis of the catheter body, a continuous electrode formed of a braided conductive mesh surrounding the flexible plastic tubing and extending over substantially all of the generally circular main region, and a support member having shape memory extending within a lumen of the flexible plastic tubing. An electrode lead wire electrically connects the continuous electrode to a source of ablation energy. The catheter further comprises means for introducing an irrigation fluid into a lumen of the flexible plastic tubing of the electrode assembly so that the fluid can pass out of the electrode assembly through the irrigation holes.

### FIELD OF THE INVENTION

The present invention relates to a catheter having a continuous braided electrode useful for creating linear lesions.

### BACKGROUND OF THE INVENTION

Atrial fibrillation is a common sustained cardiac arrhythmia and a major cause of stroke. This condition is perpetuated by reentrant wavelets propagating in an abnormal atrial-tissue substrate. Various approaches have been developed to interrupt wavelets, including surgical or catheter-mediated atriotomy. It is believed that to treat atrial fibrillation by radio-frequency ablation using a catheter, continuous linear lesions must be formed to segment the heart tissue. By segmenting the heart tissue, no electrical activity can be transmitted from one segment to another. Preferably, the segments are made too small to be able to sustain the fibrillatory process. A preferred approach for treating atrial fibrillation is by forming a linear lesion where a relatively long electrode can be held stationary in good contact with the heart wall while ablation is completed. In this way, a continuous transmural bum may be effected.

### SUMMARY OF THE INVENTION

The invention is directed to a catheter for ablating tissue comprises an elongated flexible catheter body having a continuous electrode comprising a braided conductive mesh surrounding a flexible plastic tubing.

In one embodiment, the catheter comprises a catheter body having an outer wall, proximal and distal ends, and at least one lumen extending therethrough. A tip section comprising a segment of flexible tubing having proximal and distal ends and at least one lumen therethrough is fixedly attached to the distal end of the catheter body. An electrode assembly is provided at the distal end of the tip section. The electrode assembly comprises a flexible plastic tubing and a continuous electrode formed of a braided conductive mesh surrounding the flexible plastic tubing. An electrode lead wire electrically connects the continuous electrode to a source of ablation energy.

In a particularly preferred embodiment, the catheter comprises a catheter body, a tip section at the distal end of the catheter body, and an electrode assembly at the distal end of the tip section. The electrode assembly comprises a flexible plastic tubing having an outer wall with a plurality of irrigation holes extending therethrough, a generally circular main region that is generally transverse to the axis of the catheter body, a continuous electrode formed of a braided conductive mesh surrounding the flexible plastic tubing and extending over substantially all of the generally circular main region, and a support member having shape memory extending within a lumen of the flexible plastic tubing. An electrode lead wire electrically connects the continuous electrode to a source of ablation energy. The catheter further comprises means for introducing an irrigation fluid into a lumen of the flexible plastic tubing of the electrode assembly so that, in use, the fluid can pass out of the electrode assembly through the irrigation holes.

### DESCRIPTION OF THE DRAWINGS

These and other features and advantages of the present invention will be better understood by reference to the following detailed description when considered in conjunction with the accompanying drawings wherein:

FIG. 1 is a side cross-sectional view of an embodiment of the catheter of the invention.

FIG. 2 is a side cross-sectional view of a catheter body according to the invention, including the junction between the catheter body and tip section.

FIG. 3 is a schematic perspective view of the electrode assembly according to the invention.

FIG. 4 is a side view of the electrode assembly according to the invention in a clockwise formation.

FIG. 5 is a side view of the electrode assembly according to the invention in a counterclockwise formation rotated 90° relative to the assembly depicted in FIG. 4.

FIG. 6 is a side view of the plastic tubing used to form the electrode assembly in a straight configuration.

FIG. 7A is a side view of the electrode assembly in a straight configuration and also shows the junction of the electrode assembly and the tip section.

FIG. 7B is an enlarged view of a portion of the proximal end of the electrode assembly depicted in FIG. 7A.

FIG. 7C is an enlarged view of a portion of the distal end of the electrode assembly depicted in FIG. 7A.

FIG. 8 is a cross sectional view of a portion of the catheter tip section showing one means for attaching the puller wire.

FIG. 9 is a top cross sectional views of a preferred puller wire anchor.

FIG. 10 is a side cross sectional views of the puller wire anchor of FIG. 9.

### DETAILED DESCRIPTION

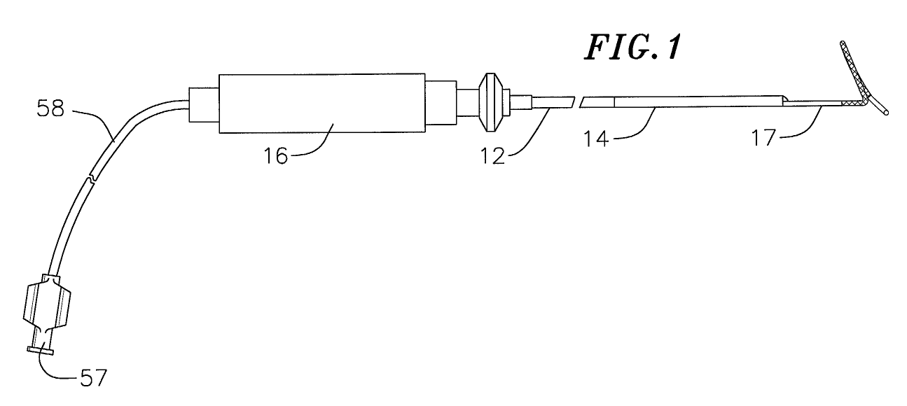

The invention is directed to a catheter having an electrode assembly comprising a braided continuous electrode. The electrode assembly preferably has a curved region that is generally transverse to the axis of the catheter and is irrigated to allow for cooling during ablation. The catheter will now be described in more detail with reference to a particularly preferred embodiment. As shown in FIG. 1, the catheter comprises an elongated catheter body 12 having proximal and distal ends, a tip section 14 at the distal end of the catheter body, a control handle 16 at the proximal end of the catheter body, with the electrode assembly 17 mounted at the distal end of the tip section 14.

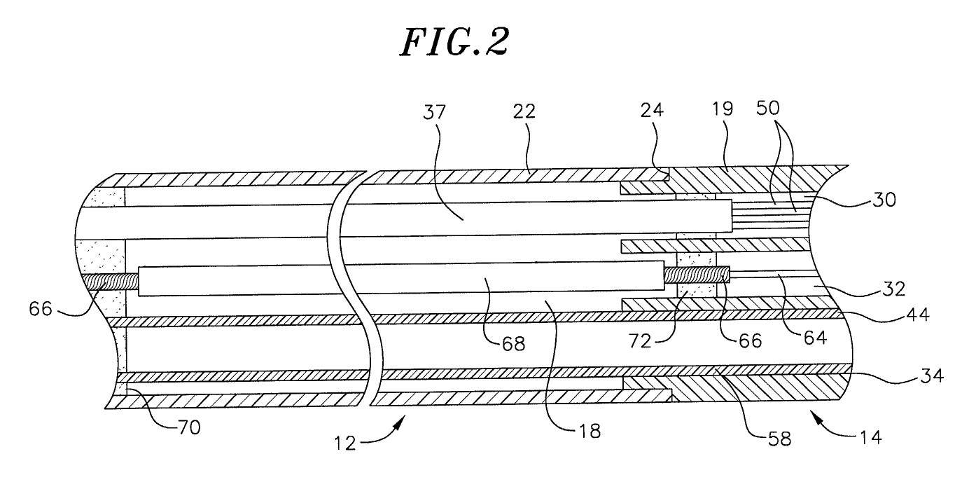

With reference to FIG. 2, the catheter body 12 comprises an elongated tubular construction having a single, axial or central lumen 18. The catheter body 12 is flexible, i.e., bendable, but substantially non-compressible along its length. The catheter body 12 can be of any suitable construction and made of any suitable material. A presently preferred construction comprises an outer wall 22 made of polyurethane or PEBAX. The outer wall 22 comprises an imbedded braided mesh of stainless steel or the like to increase torsional stiffness of the catheter body 12 so that, when the control handle 16 is rotated, the tip section 14 of the catheter will rotate in a corresponding manner.

The outer diameter of the catheter body 12 is not critical, but is preferably no more than about 8 french, more preferably 7 french. Likewise the thickness of the outer wall 22 is not critical, but is preferably thin enough so that the central lumen 18 can accommodate an infusion tube, a puller wire, lead wires, and any other wires, cables or tubes. If desired, the inner surface of the outer wall 22 is lined with a stiffening tube (not shown) to provide improved torsional stability. A particularly preferred catheter has an outer wall 22 with an outer diameter of from about 0.090 inch to about 0.94 inch and an inner diameter of from about 0.061 inch to about 0.065 inch.

The tip section 14 comprises a short section of tubing 19 having three lumens. The first lumen 30 carries electrode lead wires 50, the second lumen 32 carries a puller wire 64, and the third lumen 34 carries an infusion tube 44. The specific components within the tip section 14 are described in more detail below. The tubing 19 is made of a suitable non-toxic material that is preferably more flexible than the catheter body 12. A presently preferred material for the tubing 19 is braided polyurethane, i.e., polyurethane with an embedded mesh of braided stainless steel or the like. The size of each lumen is not critical. In a particularly preferred embodiment, the tip section 14 has an outer diameter of about 7 french (0.092 inch) and the first lumen 30 and second lumen 32 are generally about the same size, each having a diameter of from about 0.020 inch to about 0.024 inch, preferably 0.022 inch, with the third lumen 34 having a slightly larger diameter of from about 0.032 inch to about 0.038 inch, preferably 0.035 inch.

A preferred means for attaching the catheter body 12 to the tip section 14 is illustrated in FIG. 2. The proximal end of the tip section 14 comprises an outer circumferential notch 24 that receives the inner surface of the outer wall 22 of the catheter body 12. The tip section 14 and catheter body 12 are attached by glue or the like.

If desired, the catheter body 12 and tip section 14 can be formed of a single, unitary tubing, rather than being formed of two separate pieces of tubing that are joined together. Such a design might be desirable, for example, where the catheter body 12 and tip section 14 contain the same number of lumens. Accordingly, the term “tip section”, as used herein, does not require a separate piece of tubing, but instead designates the distal region of the straight, flexible tubing of the catheter.

If desired, a spacer (not shown) can be located within the catheter body between the distal end of the body and the proximal end of the tip section. The spacer provides a transition in flexibility at the junction of the catheter body and tip section, which allows this junction to bend smoothly without folding or kinking. A catheter having such a spacer is described in U.S. Pat. No. 5,964,757, the entire disclosure of which is incorporated herein by reference.

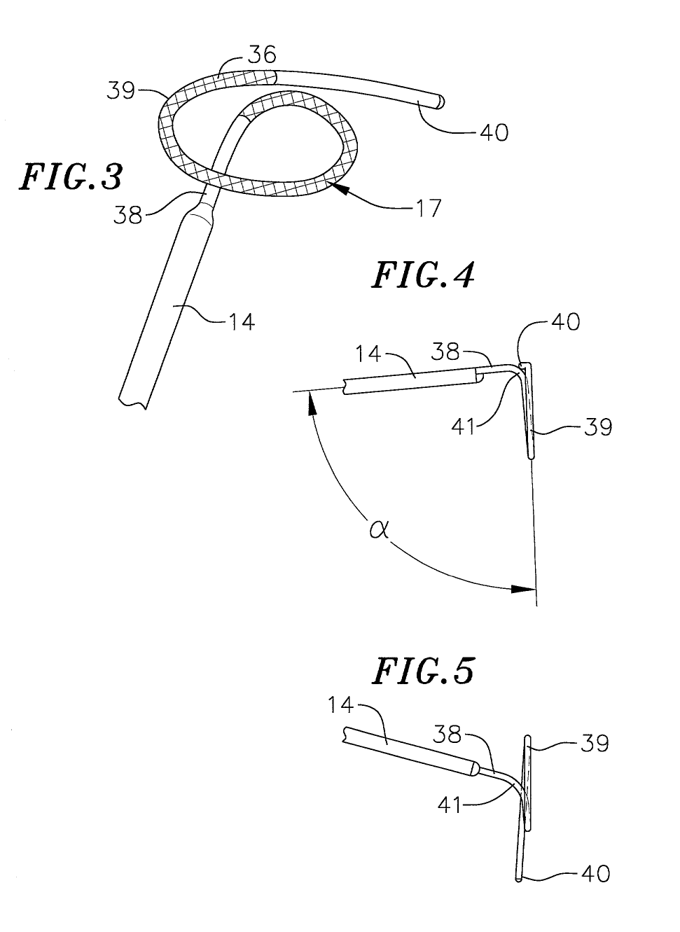

At the distal end of the tip section 14 is a generally circular electrode assembly 17, as shown in FIGS. 1 and 3 to 5. A preferred electrode assembly 17 comprises a generally straight proximal region 38, a generally circular main region 39 and a generally straight distal region 40, as best shown in FIG. 3. The proximal region 38 is mounted on the tip section 14, as described in more detail below, so that its axis is generally parallel to the axis of the tip section. The proximal region 38 preferably has an exposed length, e.g., not contained within the intermediate section 14, ranging from about 3 mm to about 12 mm, more preferably about 3 mm to about 8 mm, still more preferably about 5 mm, but can vary as desired. A curved transition region 41 joins the proximal region 38 and the main region 39.

In the depicted embodiment, the generally circular main region 39 does not form a flat circle, but is very slightly helical, and is generally transverse to the proximal region 38, tip section 14 and catheter body 12. The main region 39 generally forms a circle having an outer diameter preferably ranging to about 10 mm to about 30 mm, more preferably about 15 mm to about 25 mm. The transition region 41 of the straight proximal region 38 and generally circular main region 39 is slightly curved and formed such that, when viewed from the side with the proximal region at the top of the circular main region as shown in FIG. 4, the proximal region (along with the tip section 14) forms an angle a with the curved region preferably ranging from about 75° to about 100°. The main region 39 can curve in a clockwise direction, as shown in FIG. 4, or a counterclockwise direction, as shown in FIG. 5.

The generally circular electrode assembly 17 comprises a continuous electrode 36 along substantially all of the generally circular main region 39, which preferably has a length ranging from about 15 mm to about 35 mm. Depending on the application, the continuous electrode 36 may cover only a portion of the generally circular main region 39. The continuous electrode 36 comprises a conductive braided mesh 42 assembled over a flexible plastic tubing 45. Although the depicted embodiment comprises a substantially circular continuous electrode, the continuous electrode can take any other desired configuration, straight or curved. For some applications, the continuous electrode preferably forms a curve of at least 180°, and the curve is preferably generally transverse to the axis of the catheter.

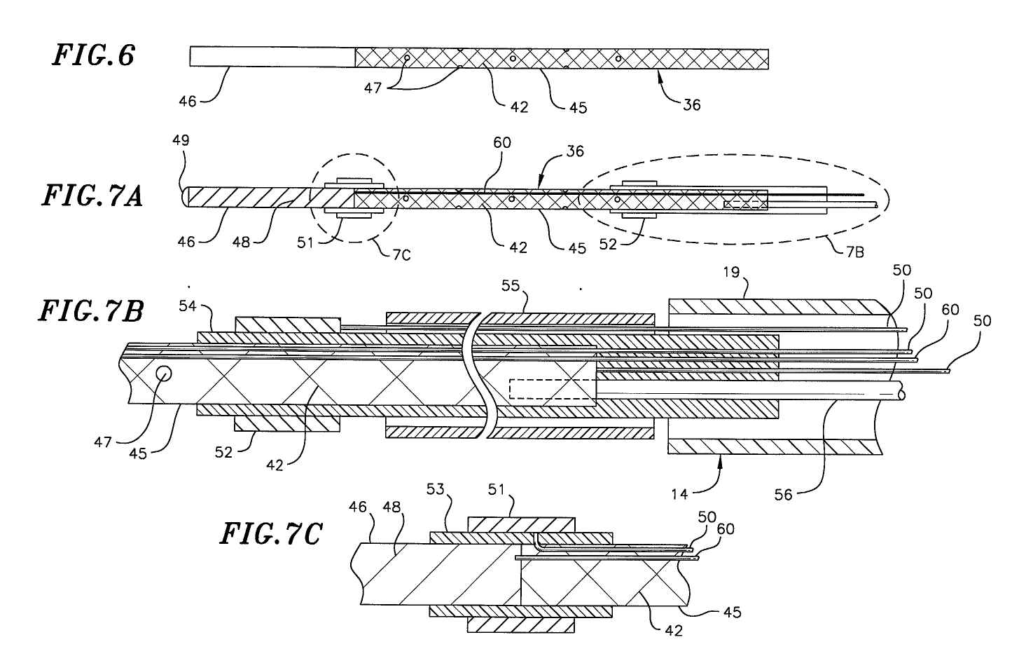

To assemble the preferred continuous electrode 36 of the invention, the braided mesh 42 is formed over the plastic, e.g., polyurethane or PEBAX, tubing 45, as shown in FIGS. 6, 7A, 7B and 7C. The plastic tubing 45 preferably has an outer diameter ranging from about 0.030 inch to about 0.080 inch, more preferably from about 0.040 inch to about 0.050 inch, with a wall thickness preferably ranging from about 0.005 inch to about 0.020, more preferably about 0.010 inch. The braided mesh 42 comprises interwoven helical members, typically twelve, sixteen or twenty-four interwoven helical members, half extending in one direction and the other half extending in the in the counter direction. The tightness or braid angle of the helical members to a line parallel with the axis of the catheter and intersecting the helical members is not critical, but is preferably about 45°. The helical members are preferably made of a conductive material having a high modulus of elasticity. Preferred helical members are made of stainless steel wire. Other methods for forming a braided mesh known in the art may be used, for example, methods typically used for forming a metal reinforcing braid within the tubing of the catheter body. During the braiding procedure, preferably the plastic tubing 45 is generally straight and has not been bent to form a generally circular arrangement, as described above. The braided mesh 42 is preferably further secured to the plastic tubing 45 with polyurethane glue or the like.

After the braided mesh 42 is formed over the plastic tubing 45, the mesh is removed from the distal region 46 of the plastic tubing, as shown in FIG. 6. In a particularly preferred embodiment, the flexible plastic tubing 42 has a length ranging from about 40 mm to about 60 mm, more preferably about 50 mm, where the distal region 46 (which is not covered by the braided mesh) has a length ranging from about 10 mm to about 20 mm, more preferably about 15 mm. Alternatively, the braided mesh 42 can be formed over only a portion of the plastic tubing 45, although it is typically easier to form the mesh over the entire tubing and thereafter remove a portion of the mesh. Once the plastic tubing 45 is fully assembled to form the generally circular electrode assembly 17, the distal region 46 of the tubing generally corresponds to the generally straight distal region 40, and the continuous electrode 36 is formed over substantially all of the generally circular main region 39 and optionally over all or a part of the generally straight proximal region 38.

Thereafter, irrigation holes 47 are formed in the plastic tubing 45 using a heated needle. It is preferred to form the irrigation holes 47 after the braided mesh 42 has been formed over the plastic tubing 45 so that the glue used to secure the mesh does not block the holes. In the depicted embodiment, the plastic tubing 45 has ten irrigation holes 47 arranged in five pairs that are generally evenly-spaced along the midsection of the plastic tubing. As can be seen in FIG. 6, the irrigation holes 47 in adjacent pairs are offset from each other a distance of about 90° about the circumference of the plastic tubing 45. By this arrangement, irrigation fluid can be more evenly distributed about the circumference of the continuous electrode 36 (e.g., braided mesh 42) during use. In a preferred embodiment, the pairs of irrigation holes 47 are longitudinally spaced apart from each other at a distance ranging from about 3 mm to about 5 mm, more preferably about 4 mm. As would be recognized by one skilled in the art, the number and arrangement of the irrigation holes 47 is not critical, but should be adequate to provide the desired amount of irrigation to the continuous electrode 36 depending on the particular application.

After the irrigation holes 47 are formed in the plastic tubing 45, the distal region 46 of the tubing is preferably modified to provide an atraumatic design that prevents the distal end of the continuous electrode 36 from penetrating tissue during use, as shown in FIG. 7A. In the depicted embodiment, the atraumatic design comprises a tightly wound coil spring 48 mounted within the distal region 46 of the plastic tubing 45 with polyurethane glue 49 or the like. The polyurethane glue 49 also acts to seal the distal end of the plastic tubing 45 (and thus the distal end of the generally circular electrode assembly 17) so that irrigation fluid entering the proximal end of the electrode assembly cannot flow out the distal end. The coil spring 48 is made, for example, of stainless steel, such as the mini guidewire commercially available from Cordis Corporation (Miami, Fla.). Additionally, if desired, the distal region 40 of the electrode assembly 17 can be formed, at least in part, of a radiopaque material to aid in the positioning of the electrode assembly 17 under fluoroscopy.

Other alternative atraumatic designs could also be provided. For example, instead of having an elongated atraumatic region, an atraumatic ball (not shown) can be formed on the distal end of the electrode assembly 17. To form the ball, the distal end of the plastic tubing 45 is covered with a short length of thick non-conductive tubing, made of polyimide, polyurethane or the like. Polyurethane adhesive or the like is applied into and around the edges of the nonconductive tubing to round off the edges of the distal end of the plastic tubing 45, thereby preventing the distal end of the plastic tubing from damaging the tissue.

The generally straight distal region 40 of the electrode assembly 17 (i.e., the distal region 46 of the plastic tubing 45 housing the coil spring 48) is preferably sufficiently long to serve as an anchor for introducing the catheter into a guiding sheath, as discussed in more detail below, because the generally circular electrode assembly must be straightened upon introduction into the sheath. Without having the generally straight distal region 40 as an anchor, the generally circular electrode assembly 17 has a tendency to pull out of the guiding sheath upon its introduction into the guiding sheath.

Preferably two ring electrodes 51 and 52 are mounted on the electrode assembly 17. A distal ring electrode 51 is mounted just distal the continuous electrode 36, and a proximal ring electrode 52 is mounted just proximal the continuous electrode. During assembly, a distal shrink sleeve 53 is formed over the distal end of the braided mesh 42 and the proximal end of the distal region 46 of the plastic tubing 45. A proximal shrink sleeve 54 is formed over a proximal region 43 of the plastic tubing 45. The distal ring electrode 51 is mounted over the distal shrink sleeve 53, and the proximal ring electrode 52 is mounted over the proximal shrink sleeve 54. Each ring electrode 51 and 52 is slid over the corresponding shrink sleeve 53 and 54 and fixed in place by glue or the like. The shrink sleeves 53 and 54 electrically isolate the ring electrodes 51 and 52 from the continuous electrode 36. The ring electrodes 51 and 52 can be made of any suitable solid conductive material, such as platinum or gold, and are preferably machined from platinum-iridium bar (90% platinum/10% iridium). Alternatively, the ring electrodes can be formed by coating the shrink sleeves 53 and 54 with an electrically conducting material, like platinum, gold and/or iridium. The coating can be applied using sputtering, ion beam deposition or an equivalent technique.

The ring electrodes 51 and 52 are each connected to a separate electrode lead wire 50. The lead wires 50 extend through the first lumen 30 of tip section 14, the central lumen 18 of the catheter body 12, and the control handle 16, and terminate at their proximal end in an input jack (not shown) mounted in the handle that may be plugged into an appropriate source of ablation energy, e.g., radio frequency energy, and/or to a monitor. The portion of the lead wires 50 extending through the central lumen 18 of the catheter body 12, control handle 16 and proximal end of the tip section 14 may be enclosed within a protective sheath 37, which can be made of any suitable material, preferably polyimide. The protective sheath 37 is preferably anchored at its distal end to the proximal end of the tip section 14 by gluing it in the first lumen 30 with polyurethane glue or the like.

The lead wires 50 are attached to the ring electrodes 51 and 52 by any conventional technique. Connection of a lead wire 50 to the distal ring electrode 51 is preferably accomplished by first making a small hole through the plastic tubing 45 and corresponding shrink sleeve 53. Such a hole can be created, for example, by inserting a needle through the tubing 45 and shrink sleeve 53 and heating the needle sufficiently to form a permanent hole. A lead wire 50 is then drawn through the hole by using a microhook or the like. The ends of the lead wire 50 are then stripped of any coating and soldered or welded to the underside of the ring electrode 51, which is then slid into position over the hole and fixed in place with polyurethane glue or the like. The lead wire 50 for the proximal ring electrode 52 preferably extends outside the plastic tubing 45 and proximal shrink sleeve 54 and is soldered to the underside of the proximal ring electrode. An outer proximal shrink sleeve 55 is then provided over the lead wire 50 connected to the proximal ring electrode to protect that lead wire.

Another lead wire 50 is electrically connected to the continuous electrode 36. Preferably the lead wire 50 is soldered to the proximal end of the braided mesh 42 prior to placement of the proximal shrink sleeve 54. The proximal shrink sleeve 42 thus acts to protect the lead wire 50 connected to the continuous electrode 36. Any other suitable method for attaching a lead wire to the continuous electrode can also be used.

A short irrigation tube 56 fluidly connects the third lumen 34 of the tip section 14 to the interior of the electrode assembly 17. The irrigation tube 56 is preferably made of polyimide, but can be made of any other suitable biocompatible material. The irrigation tube 56 provides a means for introducing irrigation fluid into the electrode assembly 17. Irrigation fluid is introduced into the third lumen 34 and irrigation tube 56 by means of an infusion tube 58, which has its distal end mounted in the proximal end of the third lumen 34 of the tip section 14 and which extends through the catheter body 12, out the proximal end of the control handle 16, and terminates in a luer hub 57 or the like at a location proximal to the control handle. In an alternative arrangement, a single lumen side arm (not shown) is fluidly connected to the central lumen 18 near the proximal end of the catheter body 12, as described in more detail in U.S. Pat. No. 6,120,476, the entire disclosure of which is incorporated herein by reference. Other means known in the art for introducing fluid into the electrode assembly could also be provided.

Preferably the generally circular shape of the electrode assembly 17 is maintained with a support member 60 that is mounted in at least a portion of the plastic tubing 45 of the electrode assembly. The support member 60 is preferably made of a material having shape-memory, i.e., that can be straightened or bent out of its original shape upon exertion of a force and is capable of substantially returning to its original shape upon removal of the force. A particularly preferred material for the support member 60 is a nickel/titanium alloy. Such alloys typically comprise about 55% nickel and 45% titanium, but may comprise from about 54% to about 57% nickel with the balance being titanium. A preferred nickel/titanium alloy is nitinol, which has excellent shape memory, together with ductility, strength, corrosion resistance, electrical resistivity and temperature stability.

In the depicted embodiment, the support member 60 has a proximal end mounted in the tip section 14 and a distal end soldered to the proximal end of the coil spring 48. Accordingly, the support member extends entirely through the generally circular main region 39 so as to define the shape of this region. By having the proximal end of the support member 60 mounted in the tip section 14, the support member acts to maintain the electrode assembly 17 on the tip section. Having a support member 60 made of a material with shape memory allows the electrode assembly 17 to be straightened when being introduced into the heart through a guiding sheath and then return to its curved formation after the guiding sheath is removed in the heart, as described in more detail below.

If desired, a temperature sensing means (not shown) can be provided for the electrode assembly 17. Any conventional temperature sensing means, e.g., a thermocouple or thermistor, may be used. One preferred temperature sensing means comprises a thermocouple formed by a wire pair. One wire of the wire pair is a copper wire, e.g., a number 38 copper wire. The other wire of the wire pair is a constantan` wire, which gives support and strength to the wire pair. The wires and can extend through the first lumen 30 in the tip section 14 along with the lead wires 50. Within the catheter body 12 the wires and can extend through the protective sheath 37, also with the lead wires 50. The wires then extend out through the control handle 16 and to the connector (not shown) in the handle, which is connectable to a temperature monitor (not shown).

A puller wire 64 is provided for deflection of the tip section 14. The puller wire 64 extends through the catheter body 12, is anchored at its proximal end to the control handle 16, and is anchored at its distal end to the tip section 14, as described in more detail below. The puller wire 64 is made of any suitable metal, such as stainless steel or Nitinol, and is preferably coated with Teflon® or the like. The coating imparts lubricity to the puller wire 64. The puller wire 64 preferably has a diameter ranging from about 0.006 to about 0.010 inch.

A compression coil 66 is situated within the catheter body 12 in surrounding relation to the puller wire 64. The compression coil 66 extends from the proximal end of the catheter body 12 to the proximal end of the tip section 14. The compression coil 66 is made of any suitable metal, preferably stainless steel. The compression coil 66 is tightly wound on itself to provide flexibility, i.e., bending, but to resist compression. The inner diameter of the compression coil 66 is preferably slightly larger than the diameter of the puller wire 64. The Teflon® coating on the puller wire 64 allows it to slide freely within the compression coil 66. If desired, particularly if the lead wires 50 are not enclosed by a protective sheath 62, the outer surface of the compression coil 66 is covered by a flexible, non-conductive sheath 68, e.g., made of polyimide tubing, to prevent contact between the compression coil and any other wires within the catheter body 12.

The compression coil 66 is anchored at its proximal end to the outer wall 22 of the catheter body 12 by proximal glue joint 70 and at its distal end to the tip section 14 by distal glue joint 72. Both glue joints 70 and 72 preferably comprise polyurethane glue or the like. The glue may be applied by means of a syringe or the like through a hole made between the outer surface of the catheter body 12 and the central lumen 18. Such a hole may be formed, for example, by a needle or the like that punctures the outer wall 22 of the catheter body 12 which is heated sufficiently to form a permanent hole. The glue is then introduced through the hole to the outer surface of the compression coil 66 and wicks around the outer circumference to form a glue joint about the entire circumference of the compression coil 66.

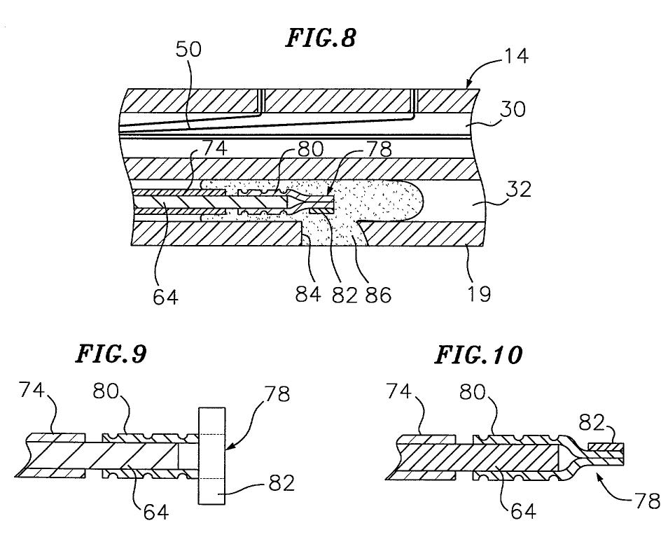

The puller wire 64 extends into the second lumen 32 of the tip section 14. Preferably the puller wire 64 is anchored at its distal end to the side of the tip section 14, as shown in FIGS. 8 to 10. A T-shaped anchor 78 is formed, which comprises a short piece of tubular stainless steel 80, e.g., hypodermic stock, which is fitted over the distal end of the puller wire 64 and crimped to fixedly secure it to the puller wire. The distal end of the tubular stainless steel 80 is fixedly attached, e.g., by welding, to a stainless steel cross-piece 82 such as stainless steel ribbon or the like. The cross-piece 82 sits in a notch 84 in a wall of the flexible tubing 19 that extends into the second lumen 32 of the tip section 14. The stainless steel cross-piece 82 is larger than the opening and, therefore, cannot be pulled through the opening. The portion of the notch 84 not filled by the cross-piece 82 is filled with glue 86 or the like, preferably a polyurethane glue, which is harder than the material of the flexible tubing 19. Rough edges, if any, of the crosspiece 82 are polished to provide a smooth, continuous surface with the outer surface of the flexible tubing 19. Within the second lumen 32 of the tip section 14, the puller wire 64 extends through a plastic, preferably Teflon®, puller wire sheath 74, which prevents the puller wire 64 from cutting into the wall of the tip section 14 when the tip section is deflected. Any other suitable technique for anchoring the puller wire 64 in the tip section 14 can also be used.

Longitudinal movement of the puller wire 64 relative to the catheter body 12, which results in deflection of the tip section 14, is accomplished by suitable manipulation of the control handle 16. Examples of suitable control handles for use in the present invention are disclosed, for example, in U.S. Pat. Nos. Re 34,502 and 5,897,529, the entire disclosures of which are incorporated herein by reference.

In use, a suitable guiding sheath is inserted into the patient. An example of a suitable guiding sheath for use in connection with the present invention is the Preface™ Braiding Guiding Sheath, commercially available from Biosense Webster (Diamond Bar, Calif.). The distal end of the sheath is guided into one of the atria. A catheter in accordance with the present invention is fed through the guiding sheath until its distal end extends out of the distal end of the guiding sheath. As the catheter is fed through the guiding sheath, the curved electrode assembly can be straightened to fit through the sheath, and it will return to its original shape upon removal of the sheath. The continuous electrode is then used to form continuous linear lesions by ablation. As used herein, a linear lesion refers to any lesion, whether curved or straight.

For treating atrial fibrillation, the linear lesion is formed between two anatomical structures in the heart and is sufficient to block a wavelet, i.e., forms a boundary for the wavelet. Anatomical structures, referred to as “atrial trigger spots”, are those regions in the heart having limited or no electrical conductivity and are described in Haissaguerre et al., “Spontaneous Initiation of Atrial Fibrillation by Ectopic Beats Originating in the Pulmonary Veins”, New England Journal of Medicine, 339:659-666 (Sep. 3, 1998), the disclosure of which is incorporated herein by reference. The linear lesions typically have a length of from about 1 cm to about 4 cm, but can be longer or shorter as necessary for a particular procedure.

During ablation, irrigation fluid may be introduced through the irrigation tube and infusion tube, into the flexible tubing of the mapping assembly, and out through the irrigation holes. The fluid acts to cool the electrode and surrounding tissue during the ablation procedure and to displace blood away from the electrode. This allows more energy to be delivered safely into the tissue to form deeper lesions and to reduce the possibility of blood coagulation. The fluid introduced through the catheter is preferably a biologically compatible fluid, such as saline.

The rate of fluid flow through the catheter may be controlled by any suitable fluid infusion pump or by pressure. A suitable infusion pump is the FLOGARD™ pump, available from Baxter. The rate of fluid flow through the catheter preferably ranges from about 0.5 ml/min to about 30 ml/min, more preferably from about 5 ml/min to about 15 ml/min. Preferably the fluid is maintained at about room temperature.

If desired, two or more puller wires can be provided to enhance the ability to manipulate the tip section. In such an embodiment, a second puller wire and a surrounding second compression coil extend through the catheter body and into an additional off-axis lumen in the tip section. The first puller wire is preferably anchored proximal to the anchor location of the second puller wire. Suitable designs of catheters having two or more puller wires, including suitable control handles for such embodiments, are described, for example, in U.S. Pat. Nos. 6,123,699, 6,171,277, and 6,183,463, and allowed U.S. patent application Ser. No. 09/157,055, filed Sep. 18, 1998, the disclosures of which are incorporated herein by reference.

The preceding description has been presented with reference to presently preferred embodiments of the invention. Workers skilled in the art and technology to which this invention pertains will appreciate that alterations and changes in the described structure may be practiced without meaningfully departing from the principal, spirit and scope of this invention.

Accordingly, the foregoing description should not be read as pertaining only to the precise structures described and illustrated in the accompanying drawings, but rather should be read consistent with and as support to the following claims which are to have their fullest and fair scope.

1. A catheter for ablating tissue comprising an elongated flexible catheter body having a continuous electrode comprising a braided conductive mesh surrounding a flexible plastic tubing.

2. A catheter according to claim 1, further comprising an electrode lead wire having a distal end electrically connected to the continuous electrode and a proximal end electrically connected to a source of ablation energy.

3. A catheter according to claim 1, wherein the continuous electrode has a length of at least about 15 mm.

4. A catheter according to claim 1, wherein the flexible tubing has an outer wall with a plurality of irrigation openings extending therethrough, and wherein the catheter further comprises means for introducing irrigation fluid into the flexible tubing, whereby, in use, fluid can pass out of the continuous electrode through the irrigation holes in the flexible tubing.

5. A catheter for ablating tissue comprising:
a catheter body having an outer wall, proximal and distal ends, and at least one lumen extending therethrough;
a tip section comprising a segment of flexible tubing having proximal and distal ends and at least one lumen therethrough, the proximal end of the tip section being fixedly attached to the distal end of the catheter body;
an electrode assembly at the distal end of the tip section, the electrode assembly comprising a flexible plastic tubing and a continuous electrode formed of a braided conductive mesh surrounding the flexible plastic tubing; and
an electrode lead wire electrically connecting the continuous electrode to a source of ablation energy.

6. A catheter according to claim 5, wherein the continuous electrode has a length of at least about 15 mm.

7. A catheter according to claim 5, wherein the electrode assembly further comprises a support member having shape memory extending within a lumen of the flexible plastic tubing.

8. A catheter according to claim 5, wherein the flexible plastic tubing has an outer wall, at least one lumen extending through the flexible plastic tubing, and a plurality of irrigation holes extending through the outer wall of the flexible plastic tubing, and wherein the catheter further comprises means for introducing an irrigation fluid into the lumen of the flexible plastic tubing of the electrode assembly, whereby, in use, fluid can pass out of the electrode assembly through the irrigation holes.

9. A catheter according to claim 5, wherein the electrode assembly comprises a curved region over which the continuous electrode extends.

10. A catheter according to claim 9, wherein the curved region is generally transverse to the axis of the catheter body.

11. A catheter according to claim 9, wherein the curved region has a curve of at least 180°.

12. A catheter according to claim 9, wherein the electrode assembly further comprises a support member having shape memory extending within a lumen of the flexible plastic tubing over at least the curved region of the electrode assembly.

13. A catheter according to claim 5, wherein the electrode assembly comprises a generally circular main region, and wherein the continuous electrode extends over at least a portion of the generally circular main region.

14. A catheter according to claim 13, wherein the generally circular main region has an outer diameter ranging to about 10 mm to about 30 mm.

15. A catheter according to claim 13, wherein the generally circular main region is generally transverse to the axis of the catheter body.

16. A catheter according to claim 15, wherein the continuous electrode extends over substantially all of the generally circular main region.

17. A catheter according to claim 16, wherein the generally circular main region has an outer diameter ranging to about 10 mm to about 30 mm.

18. A catheter according to claim 16, wherein the generally circular main region has an outer diameter ranging to about 15 mm to about 25 mm.

19. A catheter according to claim 16, wherein the electrode assembly further comprises a support member having shape memory extending within a lumen of the flexible plastic tubing over at least the generally circular main region of the electrode assembly.

20. A catheter according to claim 16, wherein the flexible plastic tubing has an outer wall, at least one lumen extending through the tubing, and a plurality of irrigation holes extending through the outer wall, and wherein the catheter further comprises means for introducing an irrigation fluid into the lumen of the tubing, whereby, in use, the fluid can pass out of the electrode assembly through the irrigation holes.

21. A catheter according to claim 13, wherein the electrode assembly further comprises a support member having shape memory extending within a lumen of the flexible plastic tubing over at least the generally circular main region of the electrode assembly.

22. A catheter according to claim 5, wherein the electrode assembly further comprises a generally straight proximal region proximal the generally circular main region, a transition region connecting the proximal region and the main region, and a generally straight distal region distal the main region.

23. A catheter according to claim 22, wherein the electrode assembly further comprises a support member having shape memory disposed within at least the main region of the electrode assembly.

24. A catheter according to claim 22, wherein the transition region is slightly curved and formed such that, when viewed from the side of the catheter with the proximal region at the top of the circular main region, the proximal region forms an angle a with the circular region ranging from about 75° to about 100°.

25. A catheter according to claim 22, wherein the generally straight distal region has an atraumatic design to prevent the distal end of the electrode assembly from penetrating tissue.

26. A catheter according to claim 22, wherein the generally straight distal region has a length ranging from about 0.25 inch to about 1.0 inch.

27. A catheter for ablating tissue comprising:
a catheter body having an outer wall, proximal and distal ends, and at least one lumen extending therethrough;
a tip section comprising a segment of flexible tubing having proximal and distal ends and at least one lumen therethrough, the proximal end of the tip section being fixedly attached to the distal end of the catheter body;
an electrode assembly at the distal end of the tip section, the electrode assembly comprising a flexible plastic tubing having an outer wall with a plurality of irrigation holes extending therethrough, a generally circular main region that is generally transverse to the axis of the catheter body, a continuous electrode formed of a braided conductive mesh surrounding the flexible plastic tubing and extending over substantially all of the generally circular main region, and a support member having shape memory extending within a lumen of the flexible plastic tubing;
an electrode lead wire electrically connecting the continuous electrode to a source of ablation energy; and
means for introducing an irrigation fluid into a lumen of the flexible plastic tubing of the electrode assembly so that the fluid can pass out of the electrode assembly through the irrigation holes.

28. A catheter according to claim 27, wherein the generally circular main region has an outer diameter ranging to about 10 mm to about 30 mm.

29. A catheter according to claim 27, wherein the generally circular main region has an outer diameter ranging to about 15 mm to about 25 mm.
